(19) United States
(12) Patent Application Publication
(10) Pub. No.: US 2024/0245508 A1
(43) Pub. Date: Jul. 25, 2024

(54) HEART VALVE IMPLANT APPARATUS

(71) Applicant: Medtronic, Inc., Minneapolis, MN (US)

(72) Inventors: Aengus O'CONNELL, Galway (IE); Timothy FARRELL, Tralee (IE); Marc ANDERSON, Barna (IE); Antonia TROTTA, Athenry (IE)

(73) Assignee: Medtronic, Inc., Minneapolis, MN (US)

(21) Appl. No.: 18/524,066

(22) Filed: Nov. 30, 2023

Related U.S. Application Data
(60) Provisional application No. 63/440,081, filed on Jan. 19, 2023.

(51) Int. Cl. A61F 2/24

(52) U.S. Cl. CPC A61F 2/2418; A61F 2/2436; A61F 2002/91566

(57) ABSTRACT

A transcatheter heart valve implant apparatus includes a frame extending along a frame axis and radially expandable between a first position and a second position. The frame includes a wire extending circumferentially around the frame axis to define a lumen. The wire includes a first peak portion including a first amplitude measured from a plane perpendicular to the frame axis, a second peak portion including a second amplitude measured from the plane, and a valley portion attaching the first peak portion and the second peak portion. The first amplitude is different than the second amplitude, and the first peak portion and the second peak portion are positioned on a first side of the plane and the valley portion is positioned on an opposing second side of the plane. A cover extends along the frame axis and covers an outer radial side of the wire opposite the lumen.

### CROSS-REFERENCE TO RELATED APPLICATIONS

This application claims the benefit of U.S. Provisional Application Ser. No. 63/440,081, filed Jan. 19, 2023.

### FIELD

The present disclosure relates generally to a heart valve implant apparatus and, more particularly, to a heart valve implant apparatus comprising a sheath with a wire.

### BACKGROUND

It is known to provide a heart valve implant apparatus for implanting a valve within a target site of the vasculature of a patient. The heart valve implant apparatus can comprise a sheath for receiving the valve. However, the sheath may lack strength and/or flexibility in one or more directions, for example, a radial direction or an axial direction. Further, manufacturing of the sheath can be costly and time-consuming.

### SUMMARY

The following presents a simplified summary of the disclosure to provide a basic understanding of some aspects described in the detailed description.

In aspects, a transcatheter heart valve implant apparatus comprises a frame extending along a frame axis and radially expandable between a first position, in which the frame comprises a first cross-sectional size, and a second position, in which the frame comprises a second cross-sectional size different than the first cross-sectional size. The frame comprises a wire extending circumferentially around the frame axis to define a lumen. The wire comprises a first peak portion comprising a first amplitude measured from a plane that is perpendicular to the frame axis and intersects the wire, a second peak portion comprising a second amplitude measured from the plane, and a valley portion attaching the first peak portion and the second peak portion. The first amplitude is different than the second amplitude. The first peak portion and the second peak portion are positioned on a first side of the plane and the valley portion is positioned on an opposing second side of the plane. A cover extends along the frame axis and covers an outer radial side of the wire opposite the lumen.

In aspects, the first peak portion is attached to a first axially-offset valley portion of the wire at a first attachment location. The second peak portion is attached to a second axially-offset valley portion of the wire at a second attachment location. The valley portion is attached to a first axially-offset peak portion of the wire at a third attachment location.

In aspects, the wire extends continuously to form the first peak portion, the second peak portion, the valley portion, the first axially-offset valley portion, the second axially-offset valley portion, and the first axially-offset peak portion.

In aspects, the frame further comprises an attachment wire extending in an axial direction substantially parallel to the frame axis. The attachment wire is attached to a plurality of different locations of the wire.

In aspects, the cover comprises one or more of an expanded polytetrafluoroethylene, a thermoplastic polyurethane, silicone, elastane, a thermoplastic polyolefin, or a thermoplastic elastomer.

In aspects, the frame comprises a jacket extending along the frame axis and covering an inner radial side of the wire.

In aspects, the jacket comprises one or more of a polyester fabric material or a polymeric material.

In aspects, the wire comprises one or more of nickel titanium or stainless steel.

In aspects, the frame comprises a braided shape with a first braided portion positioned at an inner radial side of a first wire portion of the wire and a second braided portion positioned at an outer radial side of the first wire portion, and the first braided portion positioned at the outer radial side of a second wire portion of the wire and the second braided portion positioned at the inner radial side of the second wire portion.

In aspects, an end portion of the wire is embedded in a tip of the transcatheter heart valve implant apparatus.

In aspects, a transcatheter heart valve implant apparatus comprises a frame extending along a frame axis and radially expandable between a first position, in which the frame comprises a first cross-sectional size, and a second position, in which the frame comprises a second cross-sectional size different than the first cross-sectional size. The frame comprises a wire extending circumferentially around the frame axis to define a lumen. The wire comprises a first peak portion comprising a first amplitude measured from a plane that is perpendicular to the frame axis and intersects the wire. The first peak portion is attached to a first axially-offset valley portion of the wire at a first attachment location. The wire comprises a second peak portion comprising a second amplitude measured from the plane. The second peak portion is attached to a second axially-offset valley portion of the wire at a second attachment location. The wire comprises a valley portion attaching the first peak portion and the second peak portion. The valley portion is attached to a first axially-offset peak portion of the wire at a third attachment location. The first amplitude is different than the second amplitude. The first attachment location, the second attachment location, and the third attachment location are axially-offset along the frame axis. A cover extends along the frame axis and covering an outer radial side of the wire opposite the lumen.

In aspects, the wire extends continuously to form the first peak portion, the second peak portion, the valley portion, the first axially-offset valley portion, the second axially-offset valley portion, and the first axially-offset peak portion.

In aspects, the frame further comprises an attachment wire extending in an axial direction substantially parallel to the frame axis. The attachment wire is attached to a plurality of different locations of the wire.

In aspects, the cover comprises one or more of an expanded polytetrafluoroethylene, a thermoplastic polyurethane, silicone, elastane, a thermoplastic polyolefin, or a thermoplastic elastomer.

In aspects, a jacket extends along the frame axis and covers an inner radial side of the wire. The jacket comprises one or more of a polyester fabric material or a polymeric material.

In aspects, the frame comprises a braided shape with a first braided portion positioned at an inner radial side of a first wire portion of the wire and a second braided portion positioned at an outer radial side of the first wire portion, and the first braided portion positioned at the outer radial side of a second wire portion of the wire and the second braided portion positioned at the inner radial side of the second wire portion.

In aspects, an end portion of the wire is embedded in a tip of the transcatheter heart valve implant apparatus.

In aspects, a transcatheter heart valve implant apparatus comprises a frame extending along a frame axis and radially expandable between a first position, in which the frame comprises a first cross-sectional size, and a second position, in which the frame comprises a second cross-sectional size different than the first cross-sectional size. The frame comprises a first wire portion extending circumferentially around the frame axis to define a first portion of a lumen. The first wire portion comprises a waveform shape comprising a plurality of first peak portions and a plurality of first valley portions. The frame comprises a second wire portion extending circumferentially around the frame axis to define a second portion of the lumen. The second wire portion comprises the waveform shape comprising a plurality of second peak portions and a plurality of second valley portions. The first wire portion is axially offset from the second wire portion along the frame axis. The frame comprises an attachment wire extending along the frame axis and attached at a first end to the first wire portion and at a second end to the second wire portion. A cover extends along the frame axis and covers an outer radial side of the wire opposite the lumen.

In aspects, the cover comprises one or more of an expanded polytetrafluoroethylene, a thermoplastic polyurethane, silicone, elastane, a thermoplastic polyolefin, or a thermoplastic elastomer.

In aspects, a jacket extends along the frame axis and covering an inner radial side of the wire. The jacket comprises one or more of a polyester fabric material or a polymeric material.

Additional features and advantages of the aspects disclosed herein will be set forth in the detailed description that follows, and in part will be clear to those skilled in the art from that description or recognized by practicing the aspects described herein, including the detailed description which follows, the claims, as well as the appended drawings. It is to be understood that both the foregoing general description and the following detailed description present aspects intended to provide an overview or framework for understanding the nature and character of the aspects disclosed herein. The accompanying drawings are included to provide further understanding and are incorporated into and constitute a part of this specification. The drawings illustrate various aspects of the disclosure, and together with the description explain the principles and operations thereof.

### BRIEF DESCRIPTION OF THE DRAWINGS

These and other features, aspects and advantages are better understood when the following detailed description is read with reference to the accompanying drawings, in which:

FIG. 1 schematically illustrates example aspects of an exploded transcatheter heart valve implant apparatus in accordance with aspects of the disclosure;

FIG. 2 illustrates a perspective cross-sectional view of the transcatheter heart valve implant apparatus deploying a valve in accordance with aspects of the disclosure;

FIG. 3 illustrates a perspective cross-sectional view of the transcatheter heart valve implant apparatus deploying a valve in accordance with aspects of the disclosure;

FIG. 4 illustrates a side view of example aspects of a sheath in a radially-expanded position in accordance with aspects of the disclosure;

FIG. 5 illustrates a side view of example aspects of a sheath in a radially-compressed position in accordance with aspects of the disclosure;

FIG. 6 illustrates a side view of example aspects of a sheath in a radially-expanded position with attached peak portions and valley portions in accordance with aspects of the disclosure;

FIG. 7 illustrates a side view of example aspects of the sheath of FIG. 6 in a radially-compressed position in accordance with aspects of the disclosure;

FIG. 8 illustrates a side view of example aspects of an attached peak portion and valley portion in accordance with aspects of the disclosure;

FIG. 9 illustrates a side view of example aspects of an attached peak portion and valley portion in accordance with aspects of the disclosure;

FIG. 10 illustrates a side view of example aspects of a sheath comprising an attachment wire in a radially-expanded position in accordance with aspects of the disclosure;

FIG. 11 illustrates a side view of example aspects of a sheath comprising an attachment wire in a radially-compressed position in accordance with aspects of the disclosure;

FIG. 12 illustrates a side view of example aspects of a sheath comprising attachment wires in a radially-expanded position in accordance with aspects of the disclosure.

FIG. 13 illustrates a side view of example aspects of a sheath comprising attachment wires in a radially-compressed position in accordance with aspects of the disclosure;

FIG. 14 illustrates a front side view of example aspects of a single-wire sheath in the radially-expanded position in accordance with aspects of the disclosure;

FIG. 15 illustrates a rear side view of example aspects of a single-wire sheath in a radially-expanded position in accordance with aspects of the disclosure;

FIG. 16 illustrates a side view of example aspects of a single-wire sheath with attached peak and valley portions in accordance with aspects of the disclosure;

FIG. 17 illustrates a side view of example aspects of a single-wire sheath with attached peak and valley portions in accordance with aspects of the disclosure;

FIG. 18 illustrates a cross-sectional view of example aspects of a sheath comprising a cover and a jacket in a radially-compressed position in accordance with aspects of the disclosure;

FIG. 19 illustrates a cross-sectional view of example aspects of the sheath of FIG. 18 comprising a cover and a jacket in a radially-expanded position in accordance with aspects of the disclosure;

FIG. 20 illustrates a side view of the sheath of FIG. 19 comprising a braided shape in accordance with aspects of the disclosure;

FIG. 21 illustrates a cross-sectional view of the sheath along lines 21-21 of FIG. 20 in accordance with aspects of the disclosure;

FIG. 22 illustrates a illustrates a side view of a tip of the sheath in accordance with aspects of the disclosure;

FIG. 23 illustrates a cross-sectional view of the tip of the sheath along lines 23-23 of FIG. 22 in accordance with aspects of the disclosure; and

FIG. 24 illustrates a cross-sectional view of the tip of the sheath similar to FIG. 23 in accordance with aspects of the disclosure.

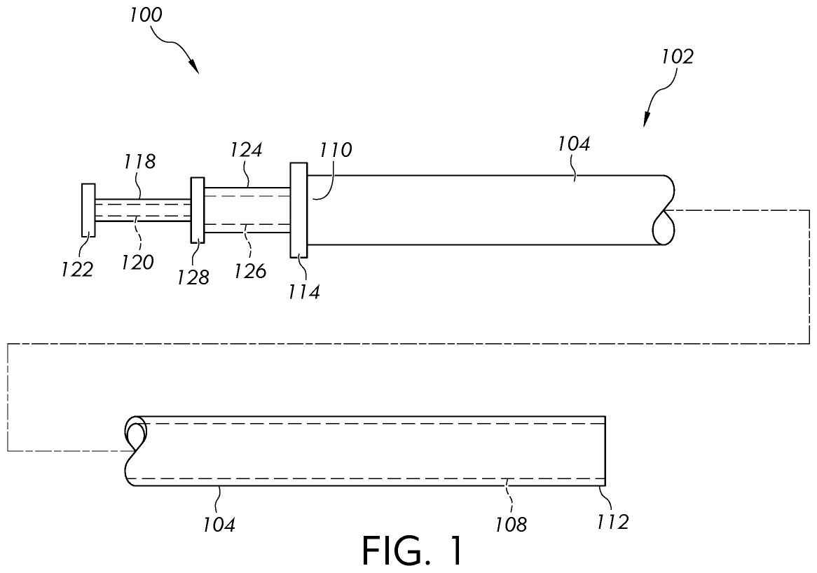

### DETAILED DESCRIPTION

Aspects will now be described more fully hereinafter with reference to the accompanying drawings in which example aspects are shown. Whenever possible, the same reference numerals are used throughout the drawings to refer to the same or like parts. However, this disclosure may be embodied in many different forms and should not be construed as limited to the aspects set forth herein.

As used herein, the term “about” means that amounts, sizes, formulations, parameters, and other quantities and characteristics are not, and need not be, exact, but may be approximate and/or larger or smaller, as desired, reflecting tolerances, conversion factors, rounding off, measurement error and the like, and other factors known to those of skill in the art. Ranges can be expressed herein as from “about” one value, and/or to “about” another value. When such a range is expressed, aspects include from the one value to the other value. Similarly, when values are expressed as approximations by use of the antecedent “about,” it will be understood that the value forms another aspect. It will be further understood that the endpoints of each of the ranges are significant both in relation to the other endpoint, and independently of the other endpoint.

Directional terms as used herein—for example up, down, right, left, front, back, top, bottom, upper, lower, etc.—are made only with reference to the figures as drawn and are not intended to imply absolute orientation.

Unless otherwise expressly stated, it is in no way intended that any methods set forth herein be construed as requiring that its steps be performed in a specific order, nor that with any apparatus, specific orientations be required. Accordingly, where a method claim does not actually recite an order to be followed by its steps, or that any apparatus claim does not actually recite an order or orientation to individual components, or it is not otherwise specifically stated in the claims or description that the steps are to be limited to a specific order, or that a specific order or orientation to components of an apparatus is not recited, it is in no way intended that an order or orientation be inferred in any respect. This holds for any possible non-express basis for interpretation, including matters of logic relative to arrangement of steps, operational flow, order of components, or orientation of components; plain meaning derived from grammatical organization or punctuation, and; the number or type of aspects described in the specification.

As used herein, the singular forms “a,” “an,” and “the” include plural references unless the context clearly dictates otherwise. Thus, for example, reference to “a” component includes aspects having two or more such components, unless the context clearly indicates otherwise.

The word “exemplary,” “example,” or various forms thereof are used herein to mean serving as an example, instance, or illustration. Any aspect or design described herein as “exemplary” or as an “example” should not be construed as preferred or advantageous over other aspects or designs. Furthermore, examples are provided solely for purposes of clarity and understanding and are not meant to limit or restrict the disclosed subject matter or relevant portions of this disclosure in any manner. It can be appreciated that a myriad of additional or alternate examples of varying scope could have been presented but have been omitted for purposes of brevity.

As used herein, the terms “comprising,” “including,” and variations thereof shall be construed as synonymous and open-ended, unless otherwise indicated. A list of elements following the transitional phrases comprising or including is a non-exclusive list, such that elements in addition to those specifically recited in the list may also be present.

The terms “substantial,” “substantially,” and variations thereof as used herein are intended to represent that a described feature is equal or approximately equal to a value or description. For example, a “substantially planar” surface is intended to denote a surface that is planar or approximately planar. Moreover, “substantially” is intended to denote that two values are equal or approximately equal. The term “substantially” may denote values within about 10% of each other, for example, within about 5% of each other, or within about 2% of each other.

Modifications may be made to the instant disclosure without departing from the scope or spirit of the claimed subject matter. Unless specified otherwise, “first,” “second,” or the like are not intended to imply a temporal aspect, a spatial aspect, an ordering, etc. Rather, such terms are merely used as identifiers, names, etc. for features, elements, items, etc. For example, a first end and a second end generally correspond to end A and end B or two different ends.

Unless otherwise indicated, the terms “distal” and “proximal” are used in the following description with respect to a position or direction relative to the treating clinician. “Distal” and “distally” are positions distant from or in a direction away from the clinician, and “proximal” and “proximally” are positions near or in a direction toward the clinician. In addition, the term “self-expanding” may be used in the following description with reference to one or more valve or stent structures of the prostheses hereof and is intended to convey that the structures are shaped or formed from a material that can be provided with a mechanical memory to return the structure from a compressed or constricted delivery configuration to an expanded deployed configuration or vice versa. Non-exhaustive exemplary self-expanding materials include stainless steel, a pseudo-elastic metal such as a nickel titanium alloy or nitinol, various polymers, or a so-called super alloy, which may have a base metal of nickel, cobalt, chromium, or other metal. Mechanical memory may be imparted to a wire or stent structure by thermal treatment to achieve a spring temper in stainless steel, for example, or to set a shape memory in a susceptible metal alloy, such as nitinol. Various polymers that can be made to have shape memory characteristics may also be suitable for use in aspects hereof to include polymers such as polynorborene, trans-polyisoprene, styrene-butadiene, and polyurethane. As well poly L-D lactic copolymer, oligo caprylactone copolymer and poly cyclo-octine can be used separately or in conjunction with other shape memory polymers.

Diseases associated with heart valves, such as those caused by damage or a defect, can include stenosis and valvular insufficiency or regurgitation. For example, valvular stenosis causes the valve to become narrowed and hardened which can prevent blood flow to a downstream heart chamber from occurring at the proper flow rate and may cause the heart to work harder to pump the blood through the diseased valve. Valvular insufficiency or regurgitation occurs when the valve does not close completely, allowing blood to flow backwards, thereby causing the heart to be less efficient. A diseased or damaged valve, which can be congenital, age-related, drug-induced, or in some instances, caused by infection, can result in an enlarged, thickened heart that loses elasticity and efficiency. Some symptoms of heart valve diseases can include weakness, shortness of breath, dizziness, fainting, palpitations, anemia and edema, and blood clots which can increase the likelihood of stroke or pulmonary embolism. Symptoms can often be severe enough to be debilitating and/or life threatening.

Heart valve prostheses have been developed for repair and replacement of diseased and/or damaged heart valves. Such heart valve prostheses can be percutaneously delivered and deployed at the site of the diseased heart valve through catheter-based delivery systems. Such heart valve prostheses generally include a frame or stent and a prosthetic valve mounted within the frame. Such heart valve prostheses are delivered in a radially compressed or crimped configuration so that the heart valve prosthesis can be advanced through the patient's vasculature. Once positioned at the treatment site, the heart valve prosthesis is expanded to engage tissue at the diseased heart valve region to, for instance, hold the heart valve prosthesis in position.

Minimally invasive percutaneous interventional procedures, including endovascular procedures, require access to the venous or arterial system. In general, it is desirable to make the smallest incision point with the shortest tissue contact time when entering the body. Small incisions and short tissue contact time generally lead to improved patient outcomes, less complications, and less trauma to the vessels or organs being accessed, as well as less trauma to the skin and tissue through which the access point is created. Access is required for various medical procedures that deliver or implant structural elements (such as heart valves, heart valve repair devices, occluders, grafts, electrical stimulators, leads, etc.) percutaneously. Some procedures employ relatively large devices that require relatively large sheaths to deliver the devices to the intended site within the body. With such procedures, access site trauma can occur, often resulting in vessel damage, excessive bleeding, increased case time, increased risk of infection, and increased hospitalization time. To reduce access trauma, physicians try to use the smallest devices possible and place the smallest sheath size. This can be problematic, however, if during the procedure the physician discovers a larger device is needed. This leads to a need to upsize the sheath, which is a lengthy procedure and leads to increased risk to the patient. Expandable sheaths can be expanded within the body and thus do not require removal to upsize.

There is a need for expandable sheath designs that are regionally or locally expansive to selectively and temporarily expand when the device is passing through a region of the sheath and to retract or recover when the device is not passing or has already passed through the sheath. Embodiments disclosed herein provide an expandable introducer sheath that may solve these and other issues that contribute to vascular trauma. Although the expandable introducer sheath disclosed herein is described with respect to percutaneous access for transcatheter heart valve repair or replacement, it should be understood that one or more features of the expandable introducer sheath may be employed alone or in combination for other medical procedures requiring percutaneous access, including but not limited to placement of stents, angioplasty, and removal of arterial or venous calcification.

Various embodiments disclosed herein relate to an introducer sheath that has a selectively expandable diameter to allow for the passage of a relatively larger device therethrough and further is configured to return to its original diameter upon passage of the device. The various embodiments may reduce damage to surrounding tissues by reducing contact with those tissues and by eliminating the need to exchange sheaths of different sizes. As a result, in comparison to known sheaths, these embodiments can reduce procedure time, vascular trauma, bleeding, and the resulting risk of infection and other complications. Although the disclosure describes a heart valve implant apparatus 100 that can be employed as an expandable introducer sheath for providing percutaneous access to the vasculature of a patient for delivering a transcatheter heart valve prosthesis, it should be understand that the disclosure is not limited to this specific application. Thus, in some aspects, the heart valve implant apparatus 100 disclosed herein may be employed in a variety of scenarios where expansion and retraction of a tubular sheath is desired, such as a delivery capsule, a delivery catheter, and a stability sheath.

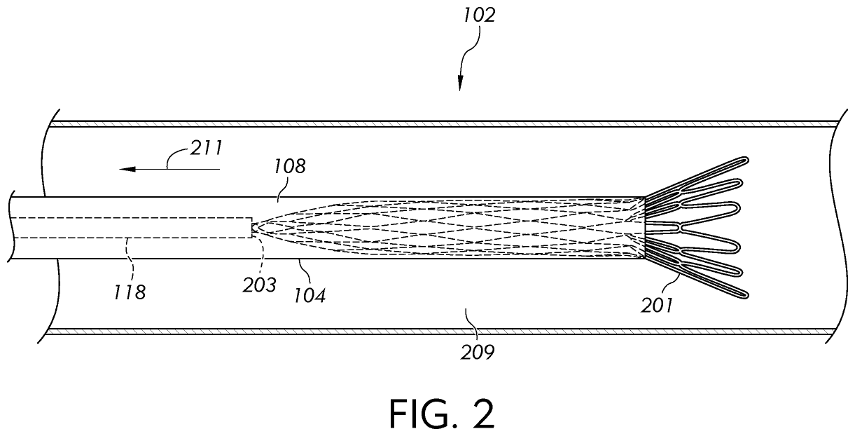

In aspects, a transcatheter heart valve implant apparatus 100 can comprise a delivery apparatus 102 that can deliver a valve to a desired location within a body of a patient, for example, a patient's heart. The delivery apparatus 102 can comprise a first sheath 104 (e.g., outer sheath, cover, etc.) that is an elongate shaft or tubular component defining a lumen 108. The first sheath 104, and, thus, the lumen 108, can extend between a proximal end 110 and a distal end 112. The first sheath 104 may be movable in an axial direction along and relative to a third sheath 124 and may extend to a proximal portion of the delivery apparatus 102, where the first sheath 104 can be controlled via an actuator, for example, a handle 114, to selectively expand a valve (e.g., valve 201 illustrated in FIG. 2). The handle 114 may comprise a push-pull actuator that may be attached or connected to the proximal end 110 of the first sheath 104. In aspects, the actuator may comprise a rotatable knob that may be attached or connected to the proximal end 110 of the first sheath 104. In this way, when the knob is rotated, the first sheath 104 can be retracted in a proximal direction to expand the valve. Alternatively, the actuator may comprise a combination of rotation and sliding to retract the first sheath 104, as described, for example, in U.S. Pat. No. 7,419,501 to Shiu et al., U.S. Patent Publication No. 2011/0257718 to Argentine, U.S. Patent Publication No. 2011/0270371 to Argentine, and U.S. Patent Publication No. 2011/0270372 to Argentine, each of which is incorporated by reference herein. Accordingly, when the actuator is operated (e.g., manually turned, pulled, etc.), the first sheath 104 can be proximally retracted over the second sheath 118 in a proximal direction.

In aspects, the delivery apparatus 102 can comprise a third sheath 124 slidingly disposed within the first sheath 104, with the second sheath 118 slidingly disposed within the third sheath 124. In aspects, the third sheath 124 can comprise one or more ribbon wires extending longitudinally through a lumen 126 of the third sheath 124. The third sheath 124 can comprise an elongate shaft or tubular component defining the lumen 126 extending between a proximal end and a distal end of the third sheath 124. The third sheath 124 can be slidingly disposed within the lumen 108 of the first sheath 104. The third sheath 124 can be movable in an axial direction along, and relative to, the second sheath 118, and may extend to a proximal portion of the delivery apparatus 102, whereupon the third sheath 124 can be controlled via an actuator, for example, a handle 128 to selectively proximally retract the one or more ribbon wires. In aspects, the handle 128 can comprise a push-pull actuator, a rotatable knob, or an actuator that uses a combination of rotation and sliding to retract the third sheath 124 as described herein with respect to the handle 114. The handle 128 may be attached or connected to a proximal end of the third sheath 124.

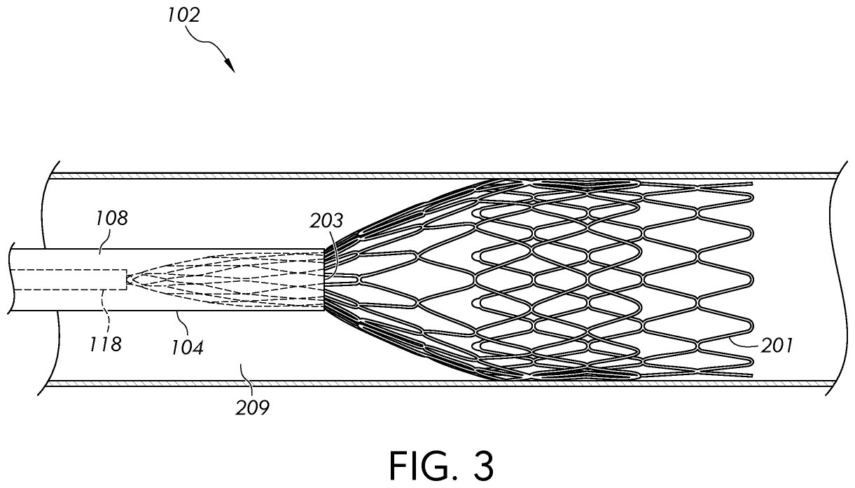

In aspects, the second sheath 118 can comprise an elongate shaft or tubular component that defines a lumen 120 extending from a proximal end to a distal end of the second sheath 118. The lumen 120 of the second sheath 118 can be sized to slidingly receive a plunger with a dilator tip at a distal end thereof. The plunger can define a guidewire lumen such that the delivery apparatus 102, comprising the plunger extending therethrough, may be advanced over a guidewire to assist in tracking the delivery apparatus 102 to a target site within the vasculature. The second sheath 118 can be slidingly disposed within the lumen 126 of the third sheath 124. In aspects, a distal end of the second sheath 118 can be coupled to a proximal end of the valve (e.g., illustrated in FIGS. 2-3). For example, the distal end of the second sheath 118 can be releasably coupled to the proximal end of the valve such that the valve may be selectively detached from the delivery apparatus 102. The second sheath 118 may be movable in an axial direction along, and relative to, the first sheath 104 and may extend to a proximal end of the delivery apparatus 102. The second sheath 118 may be controlled via an actuator, such as a handle 122, to selectively proximally retract the valve. The handle 122 can comprise, for example, a push-pull actuator, a rotatable knob, or an actuator that uses a combination of rotation and sliding to retract the second sheath 118 as described herein with respect to handle 114 and is attached or connected to proximal end of the second sheath 118.

Referring to FIG. 2, in aspects, a valve prosthesis 201 (e.g., hereinafter “valve 201” or “valve prosthesis 201”) may be held within the lumen 108 when the valve 201 is in a radially-compressed and retracted position. The valve prosthesis 201 can comprise, for example, one or more of a stent structure, scaffold structure, balloon-expandable valve, self-expanding valve, or other prostheses comprising valve leaflets attached to an interior of the valve 201. To couple the valve 201 to the delivery apparatus 102, a distal end 203 of the second sheath 118 can be coupled to a proximal end of the valve 201. In this way, the valve 201 may be releasably coupled to the second sheath 118 while the valve 201 is radially-compressed and positioned in the lumen 108. The delivery apparatus 102 can be moved within a vasculature 209 to a desired location. In aspects, upon reaching the desired location, the valve 201 can be deployed and decoupled from the second sheath 118. For example, the first sheath 104 can be retracted as illustrated by directional arrow 211 such that the valve 201 can be partially deployed. In aspects, one or more flat wires may be provided within the delivery apparatus 102 to facilitate deployment of the valve 201 and retraction of the first sheath 104. Retraction of the first sheath 104 can continue, as illustrated in FIG. 3, until the valve 201 is completely outside of the lumen 108 and fully expanded (e.g., full expansion illustrated in FIG. 3). In aspects, one or more flat wires can control deployment of the valve 201 and assist in positioning and/or recapture of the valve 201 if necessary. Upon reaching a desired position, the valve 201 may be separated from the delivery apparatus 102.

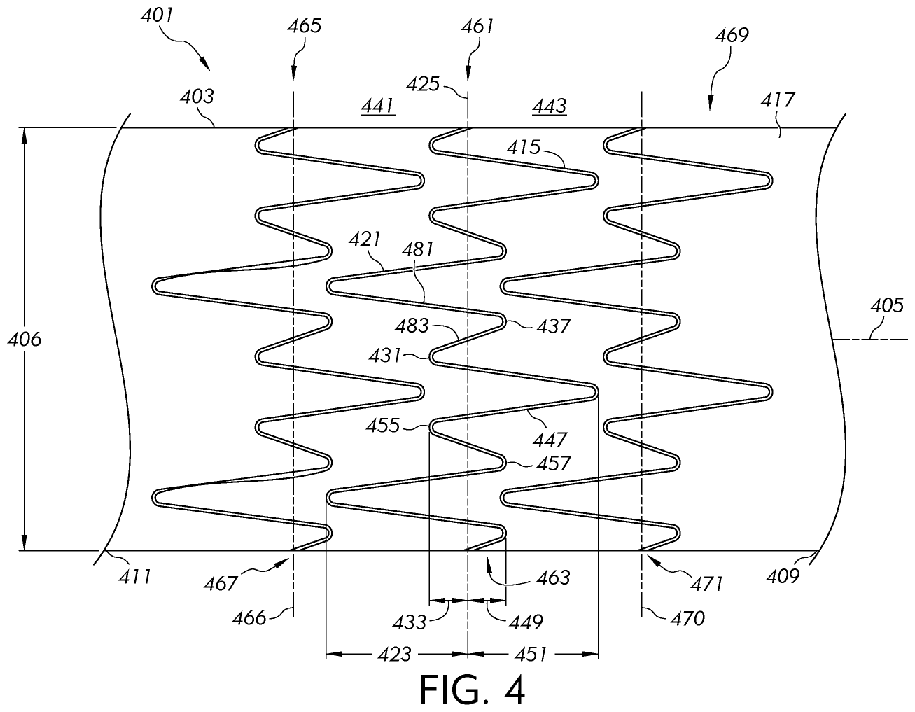

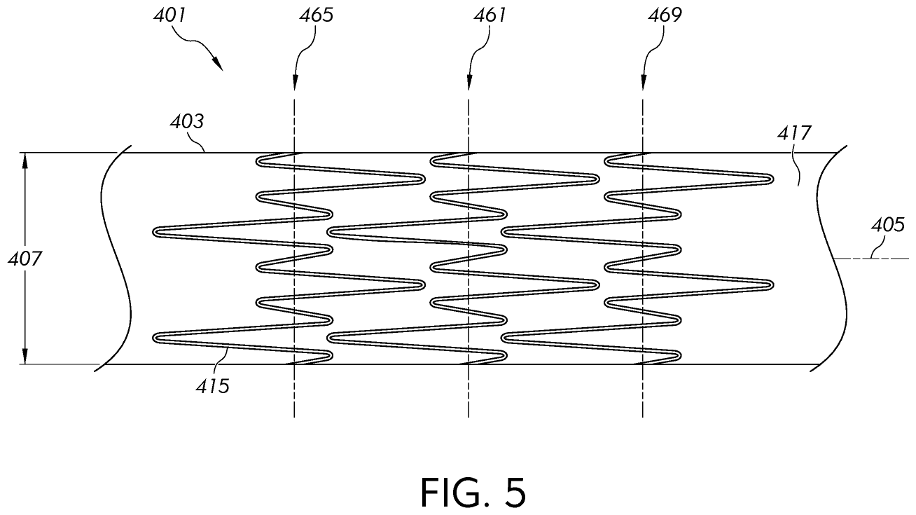

FIG. 4 illustrates a side perspective view of a sheath 401 that can be used within the delivery apparatus 102. For example, one or more of the first sheath 104, the second sheath 118, or the third sheath 124 may be substantially identical to the sheath 401 illustrated and described herein. In aspects, the sheath 401 can comprise a frame 403 that can extend along a frame axis 405. The frame 403 can be radially-expandable between a first position (e.g., illustrated in FIG. 4), in which the frame 403 comprises a first cross-sectional size 406, and a second position (e.g., illustrated in FIG. 5), in which the frame 403 comprises a second cross-sectional size 407 different than the first cross-sectional size 406. In aspects, the second cross-sectional size 407 may be less than the first cross-sectional size 406, such that the first position is a radially-expanded position and the second position is a radially-contracted position. The frame 403 may comprise a material comprising a mechanical memory that may maintain the frame 403 in a linear, axially-extending configuration, for example, by extending axially along the frame axis 405. The frame 403 can move from the axially-extending configuration to a non-axial bent or curved configuration, for example, as the sheath 401 moves within the vasculature. Due to the mechanical memory, the frame 403 can return to the axially-extending configuration in the absence of forces acting upon the frame 403. The frame 403 can extend between a distal end 409 and a proximal end 411.

The frame 403 can comprise a wire 415 that extends circumferentially around the frame axis 405 to define a lumen 417. The wire 415 can comprise several types of materials, for example, one or more of nickel titanium (e.g., nitinol) or stainless steel. The lumen 417 is substantially hollow and can be sized to receive the valve 201, such that the frame 403 can hold and maintain the valve 201 in a radially-compressed position. In aspects, the wire 415 can comprise a waveform shape with one or more peak portions and one or more valley portions. For example, the wire 415 can comprise a first peak portion 421 comprising a first amplitude 423 measured from a plane 425 that is perpendicular to the frame axis 405, with the plane 425 intersecting the wire 415. The wire 415 can comprise a second peak portion 431 comprising a second amplitude 433 measured from the plane 425. The wire 415 can comprise a valley portion 437 attaching the first peak portion 421 and the second peak portion 431. In aspects, the first peak portion 421 and the second peak portion 431 may be positioned on a first side 441 of the plane 425 and the valley portion 437 may be positioned on an opposing second side 443 of the plane 425. In aspects, the first amplitude 423 may be different than the second amplitude 433, for example, with the first amplitude 423 greater than the second amplitude 433.

The peak portions 421, 431 can comprise maximum distances from the plane 425 on the first side 441 of the plane 425. For example, referring to the first peak portion 421, at a location along the wire 415 moving toward the first peak portion 421, the wire 415 may gradually increase in distance (or amplitude) from the plane 425, whereupon a maximum distance (e.g., the first amplitude 423) is reached at the first peak portion 421, followed by a gradually decreasing distance (or amplitude) from the plane 425. Likewise, referring to the second peak portion 431, at a location along the wire 415 moving toward the second peak portion 431, the wire 415 may gradually increase in distance (or amplitude) from the plane 425, whereupon a maximum distance (e.g., the second amplitude 433) is reached at the second peak portion 431, followed by a gradually decreasing distance (or amplitude) from the plane 425. The valley portion 437 can comprise a maximum distance from the plane 425 on the second side 443 of the plane 425. For example, at a location along the wire 415 moving toward the valley portion 437, the wire 415 may gradually increase in distance (or amplitude) from the plane 425, whereupon a maximum distance (e.g., a third amplitude 449) is reached at the valley portion 437, followed by a gradually decreasing distance (or amplitude) from the plane 425.

In aspects, the wire 415 is not limited to comprising the first peak portion 421, the second peak portion 431, and the valley portion 437. Rather, the wire 415 can comprise a second valley portion 447 connected to the second peak portion 431, such that the second peak portion 431 is positioned between, and connected to, the valley portion 437 and the second valley portion 447, with the valley portion 437 and the second valley portion 447 positioned on the second side 443 of the plane 425. In aspects, the valley portion 437 can comprise the third amplitude 449 and the second valley portion 447 can comprise a fourth amplitude 451, wherein the third amplitude 449 is different than the fourth amplitude 451. For example, the third amplitude 449 may be less than the fourth amplitude 451. In aspects, the first amplitude 423 can substantially match the third amplitude 449, and the second amplitude 433 can substantially match the fourth amplitude 451.

The wire 415 can be arranged such that the peak portions 421, 431 and the valley portions 437, 447 can extend circumferentially around the frame axis 405. For example, the first peak portion 421 comprising the first amplitude 423 can be connected to, and adjacent to, the valley portion 437 comprising the third amplitude 449. The valley portion 437 can be connected to, and adjacent to, the second peak portion 431 comprising the second amplitude 433, with the valley portion 437 connecting, and positioned between, the first peak portion 421 and the second peak portion 431. The second peak portion 431 can be connected to, and adjacent to, the second valley portion 447 comprising the fourth amplitude 451, with the second peak portion 431 connecting, and positioned between, the valley portion 437 and the second valley portion 447. The second valley portion 447, opposite the second peak portion 431, can be connected to a third peak portion 455 which may be substantially identical in shape and amplitude to the second peak portion 431. The third peak portion 455, opposite the second valley portion 447, can be connected to a third valley portion 457, which may be substantially identical in shape and amplitude to the valley portion 437. The wire 415 may then repeat the arrangement of peak portions and valley portions.

In aspects, the wire 415 may comprise a plurality of levels of peak portions and valley portions. For example, the plane 425 can intersect a first level 461 of the wire 415, wherein the first level 461 comprises the peak portions 421, 431, 455 and the valley portions 437, 447, 457, along with additional peak portions (e.g., substantially identical in shape, size, and arrangement to the peak portions 421, 431, 455) and additional valley portions (e.g., substantially identical in shape, size, and arrangement to the valley portions 437, 447, 457). The first level 461 can extend circumferentially around the frame axis 405, such that the first level 461 is located at a first axial location 463 along the frame axis 405. In aspects, the wire 415 can comprise a second level 465, wherein the second level 465 comprises peak portions and valley portions substantially identical to the peak portions 421, 431, 455 and the valley portions 437, 447, 457 described herein. The second level 465 may extend circumferentially around the frame axis 405 and axially offset from the first level 461, such that the second level 465 is located at a second axial location 467, different than the first axial location 463, along the frame axis 405. A second plane 466 that is perpendicular to the frame axis 405 and parallel to the plane 425 can intersect the second level 465, with the second plane 466 substantially parallel to the plane 425. In aspects, the wire 415 can comprise a third level 469, wherein the third level 469 comprises peak portions and valley portions substantially identical to the peak portions 421, 431, 455 and the valley portions 437, 447, 457 described herein. The third level 469 may extend circumferentially around the frame axis 405 and axially offset from the first level 461 and the second level 465, such that the third level 469 is located at a third axial location 471, different than the first axial location 463 and the second axial location 467, along the frame axis 405. A third plane 470 that is perpendicular to the frame axis 405 and parallel to the planes 425, 466 can intersect the third level 469, with the third plane 470 substantially parallel to the plane 425 and the second plane 466.

In aspects, the first axial location 463 is positioned between the second axial location 467 and the third axial location 471. In this way, the first level 461 may be positioned between the second level 465 and the third level 469. A first distance may separate the first axial location 463 from the second axial location 467, and a second distance may separate the first axial location 463 from the third axial location 471. In aspects, the first distance may be substantially equal to the second distance, though, in other aspects, the first distance may be different than the second distance. Accordingly, the levels 461, 465, 469 can be spaced apart and arranged along the frame axis 405 with additional levels located beyond the second level 465 and beyond the third level 469. While the wire 415 is illustrated as comprising the levels 461, 465, 469 that are intersected by planes 425, 466, 470 perpendicular to the frame axis 405, the arrangement 415 of the wire 415 is not so limited. Rather, in aspects, the wire 415 can comprise a helical shape winding around the frame axis 405 in which lines tangent to the wire 415 are at a constant angle to the frame axis 405. That is, the first level 461 can be intersected by the plane 425, wherein the plane 425 is non-perpendicular to the frame axis 405. Likewise, the second level 465 can be intersected by the second plane 466, wherein the second plane 466 is non-perpendicular to the frame axis 405 and parallel to the plane 425. The third level 469 can be intersected by the third plane 470, wherein the third plane 470 is non-perpendicular to the frame axis 405 and parallel to the second plane 466. In these examples, and as illustrated in FIG. 4, in aspects, the first level 461 may not contact the second level 465 or the third level 469.

The wire 415 may comprise one or more intermediate wire portions. For example, a first intermediate portion 481 may extend between the first peak portion 421 and the valley portion 437, and a second intermediate portion 483 may extend between the valley portion 437 and the second peak portion 431. In aspects, as illustrated in FIG. 4, the first intermediate portion 481 and the second intermediate portion 483 may be angled relative to one another, with the first intermediate portion 481 and the second intermediate portion 483 converging to form the valley portion 437. In this way, the first intermediate portion 481 may be non-parallel to the second intermediate portion 483, and both intermediate portions 481, 483 may be non-parallel with the frame axis 405. Other intermediate portions of the wire 415 may be arranged similarly to the intermediate portions 481, 483. The wire 415 is not so limited, however, and in aspects, the first intermediate portion 481 and the second intermediate portion 483 (e.g., and other intermediate portions extending between peak portions and valley portions) may be substantially parallel to one another, and substantially parallel with the frame axis 405.

FIG. 5 illustrates the sheath 401 in the second position, in which the frame 403 has radially-contracted from the first position (e.g., illustrated in FIG. 4). In the first position, the frame 403 comprises the first cross-sectional size 406, and in the second position, the frame 403 comprises the second cross-sectional size 407 that is less than the first cross-sectional size 406. In aspects, when a valve or other structure is positioned within the lumen 417, the sheath 401 may radially-expand to the first cross-sectional size 406 of the first position. After the valve or other structure has been deployed and removed from the lumen 417, the sheath 401 may radially-contract to the second cross-sectional size 407 of the second position.

The radial-expansion and contraction may be facilitated by characteristics of the wire 415. For example, a material of the wire (e.g., nickel titanium, stainless steel, etc.) can retain a shape memory that biases the sheath 401 to the second position comprising the smaller second cross-sectional size 407. In addition, the waveform shape of the wire 415 can allow for expansion and contraction of the sheath 401, both in the radial and axial directions, due to a circumference of the sheath 401 being less than a length of the wire 415 at one of the levels 461, 465, 469. For example, the levels 461, 465, 469 may be spaced apart but may overlap, such that a plane perpendicular to the frame axis 405 can intersect the first level 461 and the second level 465, and another plane, perpendicular to the frame axis 405, can intersect the first level 461 and the second level 465. This overlapping waveform configuration of the wire 415 can provide for additional strength to the sheath 401. In addition, as described below, the sheath 401 may comprise an inner radial cover and/or an outer radial cover to further facilitate expansion and contraction of the sheath 401. In addition to providing for radial-expansion and contraction, the sheath 401 may exhibit resistance to kinking when moving within the vasculature of a patient. For example, as the sheath 401 moves, the sheath 401 may be exposed to forces that cause the sheath 401 to bend, curve, or otherwise extend non-linearly. The avoidance of kinking of the sheath 401 is beneficial to limit collapse of the sheath 401 during movement. Because the wire 415 comprises the waveform shape with alternating peak portions and valley portions, the peak portions and the valley portions may interlock, or overlap, while bending. The interlocking and overlapping nature of the wire 415 can function to increase radial strength of the sheath 401 and maintain the diameter of the lumen 417 even in tortuous anatomy.

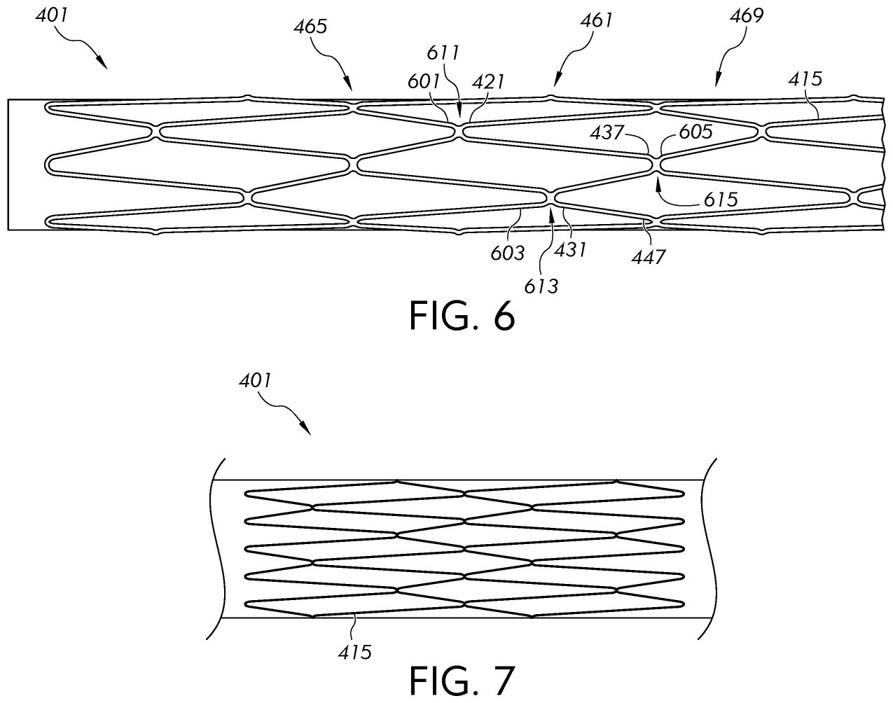

FIG. 6 illustrates additional aspects of the sheath 401, with the sheath 401 in the radially-expanded first position. In aspects, the sheath 401 may comprise some identical features as illustrated and described in FIGS. 4-5, for example, the wire 415 comprising the peak portions 421, 431, 455 and the valley portions 437, 447, 457, the levels 461, 465, 469, etc. However, as illustrated in FIGS. 4-5, the peak portions and valley portions of one level may not be attached to peak portions and valley portions of an adjacent level. In contrast, as illustrated in FIG. 6, the peak portions of the first level 461 may be attached to the valley portions of the adjacent second level 465, and the valley portions of the first level 461 may be attached to the peak portions of the adjacent third level 469.

For example, the sheath 401 can comprise the peak portions 421, 431, 455 and the valley portions 437, 447, 457 arranged in the first level 461. In aspects, the arrangement of the peak portions and the valley portions in the second level 465 may be substantially identical to the peak portions 421, 431, 455 and the valley portions 437, 447, 457 of the first level 461, with the wire 415 comprising a first axially-offset valley portion 601 and a second axially-offset valley portion 603 within the second level 465. In aspects, the arrangement of the peak portions and the valley portions in the third level 469 may be substantially identical to the peak portions 421, 431, 455 and the valley portions 437, 447, 457 of the first level 461, with the wire 415 comprising a first axially-offset peak portion 605 within the third level 469. In aspects, the first peak portion 421 can be attached to the first axially-offset valley portion 601 of the wire 415 at a first attachment location 611, wherein the first axially-offset valley portion 601 may be substantially identical in shape and dimension to the valley portion 437. In aspects, the second peak portion 431 can be attached to the second axially-offset valley portion 603 of the wire 415 at a second attachment location 613. Accordingly, the first level 461 can be attached to the second level 465 due to the attachment of peaks of the first level 461 to valleys of the second level 465.

In aspects, the valley portion 437 can be attached to the first axially-offset peak portion 605 of the wire 415 at a third attachment location 615, wherein the first axially-offset peak portion 605 may be substantially identical in shape and dimension to the first peak portion 421. Accordingly, the first level 461 can be attached to the third level 469 due to the attachment of valleys of the first level 461 to peaks of the third level 469. In aspects, the first attachment location 611, the second attachment location 613, and the third attachment location 615 may be axially-offset along the frame axis 405. In aspects, some, or all of the peaks and valleys of adjacent levels can be attached. FIG. 7 illustrates the sheath 401 in the radially-contracted second position. As illustrated, the sheath 401 can move between the radially-expanded first position and the radially-contracted second position while the peaks and valleys of adjacent rows remain attached. By attaching the levels 461, 465, 469 at the peak portions and valley portions, the sheath 401 can exhibit increased radial and axial strength and also improved flexibility.

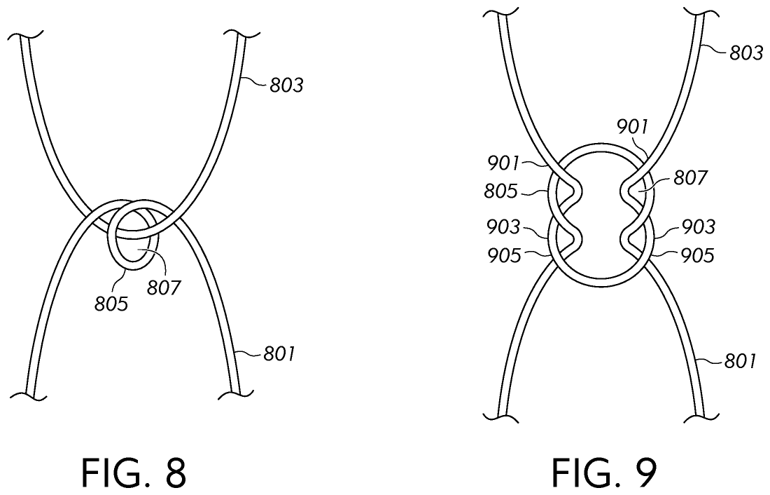

FIG. 8 illustrates example aspects of attaching a peak portion 801 and a valley portion 803, for example, in aspects in which portions of the levels 461, 465, 469 are attached at the peak portions and valley portions. In aspects, the peak portion 801 may be substantially identical to one or more of the peak portions 421, 431, 455, 605 described herein, and the valley portion 803 may be substantially identical to one or more of the valley portions 437, 447, 457, 601 described herein. In aspects, an end of the peak portion 801 can overlap to form a wire loop 805 defining an opening 807. The opening 807 may be circumferentially bounded by the wire 415, and the valley portion 803 may extend through the opening 807. As such, the peak portion 801 and the valley portion 803 can be attached. A benefit of the attachment of the peak portion 801 and the valley portion 803 illustrated in FIG. 8 is that additional attachment materials or steps (e.g., adhesives, laser, suturing, etc.) may not be needed.

FIG. 9 illustrates another example aspect of attaching the peak portion 801 and the valley portion 803. For example, the peak portion 801 can be formed into the wire loop 805 to define the opening 807. The valley portion 803 can comprise first overlapping portions 901 that pass over the wire loop 805 and extend through the opening 807 from a first side to a second side, underlying portions 903 that extend under the wire loop 805, and second overlapping portions 905 that extend through the opening 807 from the second side to the first side. In this way, the underlying portions 903 can attach the first overlapping portions 901 and the second overlapping portions 905, with the overlapping portions 901, 905 passing through the opening 807. As such, the peak portion 801 and the valley portion 803 can be attached. As with the aspect of FIG. 8, a benefit of the attachment of the peak portion 801 and the valley portion 803 illustrated in FIG. 9 is that additional attachment materials or steps (e.g., adhesives, laser, suturing, etc.) may not be needed.

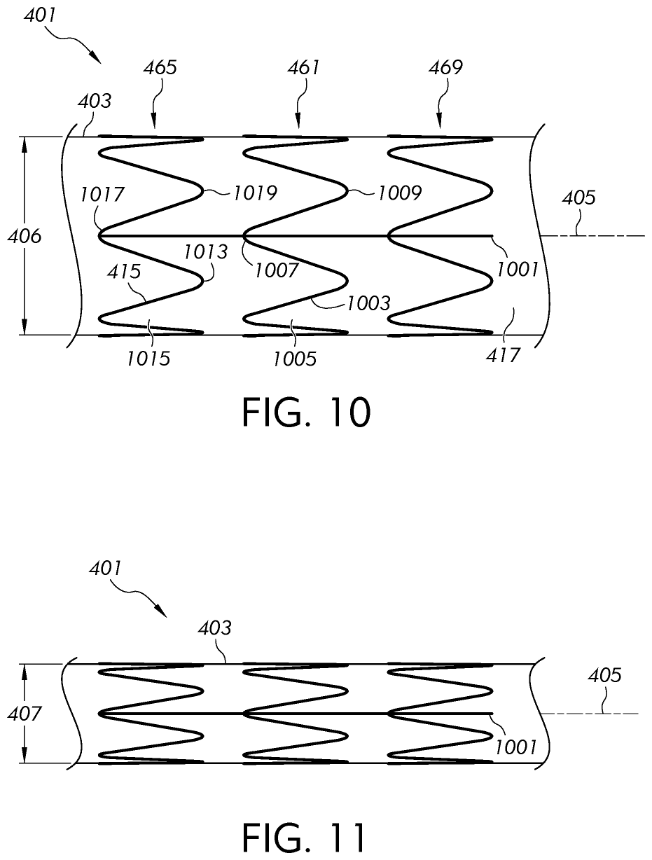

FIG. 10 illustrates another example aspect of attaching portions of the wire 415. In aspects, the frame 403 can comprise an attachment wire 1001 extending in an axial direction substantially parallel to the frame axis 405, with the attachment wire 1001 attached to a plurality of different locations of the wire 415. For example, in aspects, the attachment wire 1001 can extend substantially linearly and may comprise a shape memory to remain in the substantially-linear orientation. The attachment wire 1001 can be attached to one or more locations of the wire 415. For example, the wire 415 can comprise the plurality of levels 461, 465, 469, etc. In aspects, a first wire portion 1003 of the frame 403 can form the first level 461, with the first wire portion 1003 extending circumferentially around the frame axis 405 to define a first portion 1005 of the lumen 417. The first wire portion 1003 can comprise a waveform shape comprising a plurality of first peak portions 1007 and a plurality of first valley portions 1009. In aspects, for example, as illustrated in FIG. 10, the waveform shape can comprise a continuous wave (e.g., a sinewave) comprising a constant amplitude and frequency. Alternatively, the waveform shape can comprise the shape illustrated in FIGS. 4-9, wherein the first peak portions 1007 are substantially identical to the peak portions 421, 431, 455 and the first valley portions 1009 are substantially identical to the valley portions 437, 447, 457, with a non-constant amplitude.

In aspects, a second wire portion 1013 of the frame 403 can form the second level 465, with the second wire portion 1013 extending circumferentially around the frame axis 405 to define a second portion 1015 of the lumen 417. The second wire portion 1013 can comprise a waveform shape comprising a plurality of second peak portions 1017 and a plurality of second valley portions 1019. In aspects, the waveform shape can comprise a continuous wave or the shape illustrated in FIGS. 4-9, wherein the second peak portions 1017 are substantially identical to the peak portions 421, 431, 455 and the second valley portions 1019 are substantially identical to the valley portions 437, 447, 457. In aspects, the first wire portion 1003 may be substantially identical in size and shape to the second wire portion 1013, with the first wire portion 1003 axially offset from the second wire portion 1013 along the frame axis 405. In addition, the frame 403 can comprise additional wire portions substantially identical to the first wire portion 1003 and the second wire portion 1013 that are axially offset from the first wire portion 1003 and the second wire portion 1013.

The attachment wire 1001 can extend along the frame axis 405 and may be attached at one end or location to the first wire portion 1003 and at another end or location to the second wire portion 1013. For example, the first peak portions 1007 can be aligned with the second peak portions 1017, and the first valley portions 1009 can be aligned with the second valley portions 1019. In this way, an axis extending substantially parallel to the frame axis 405 can intersect a peak portion of the first peak portions 1007 and a peak portion of the second peak portions 1017, and a different axis extending substantially parallel to the frame axis 405 can intersect a valley portion of the first valley portions 1009 and a valley portion of the second valley portions 1019.

In aspects, the attachment wire 1001 can be attached to some or all portions of the levels or wire portions 1003, 1013 of the frame 403. That is, the attachment wire 1001 can extend substantially parallel to the frame axis 405 and may be attached to one of the peak portions of the first peak portions 1007, one of the peak portions of the second peak portions 1017, etc. In the alternative, the attachment wire 1001 can extend substantially parallel to the frame axis 405 and may be attached to one of the valley portions of the first valley portions 1009, one of the valley portions of the second valley portions 1019 etc.

FIG. 10 illustrates the frame 403 in the radially-expanded first position, in which the frame 403 comprises the first cross-sectional size 406, while FIG. 11 illustrates the frame 403 in the radially-contracted second position, in which the frame 403 comprises the radially-contracted second cross-sectional size 407. The attachment wire 1001 can provide column strength to the frame 403 which can assist in insertion of the sheath 401 and movement of the sheath 401 within the vasculature of a patient. The column strength is the strength of the sheath 401 along the frame axis 405 and resistance to unintended collapse or snaking of the sheath 401 as the sheath 401 is moved within the vasculature. In aspects, the attachment wire 1001 is flexible to allow for bending or curving of the sheath 401 as the sheath 401 follows a tortuous path within the vasculature, while limiting the sheath 401 from axially collapsing in a direction along the frame axis 405.

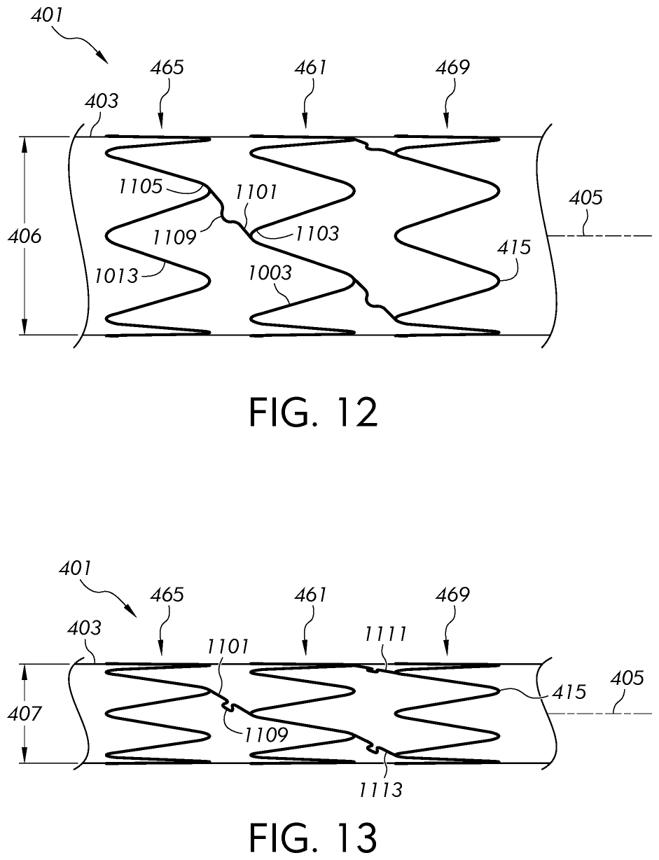

FIG. 12 illustrates another example aspect of attaching portions of the wire 415. For example, instead of the frame 403 comprising the single attachment wire 1001 extending linearly and parallel to the frame axis 405, the frame 403 can comprise a plurality of attachment wires that can attach the different wire portions of the frame 403. For example, the frame 403 can comprise a first attachment wire 1101 that can attach a peak portion 1103 of the first wire portion 1003 to a valley portion 1105 of the second wire portion 1013. In aspects, the first attachment wire 1101 can comprise a first expansion feature 1109 that can facilitate radial and/or axial expansion and contraction of the frame 403. Due to the peak portions 1103 of the first wire portion 1003 being circumferentially offset from the valley portions 1005 of the second wire portion 1013, the first attachment wire 1101 may be non-parallel to the frame axis 405.

FIG. 12 illustrates the frame 403 in the radially-expanded first position, in which the frame 403 comprises the first cross-sectional size 406, while FIG. 13 illustrates the frame 403 in the radially-contracted second position, in which the frame 403 comprises the second cross-sectional size 407. In the radially-contracted second position of FIG. 13, the first expansion feature 1109 may comprise excess wire material such that the first attachment wire 1101 may not extend linearly between the peak portion 1103 and the valley portion 1105. Rather, the first expansion feature 1109 can form a notch or other non-linear extension of the first attachment wire 1101 at a location between the peak portion 1103 and the valley portion 1105. In aspects, other attachment wires 1111, 1113 may be provided to attach other peak portions and valley portions, with the other attachment wires 1111, 1113 substantially identical in structure and function to the first attachment wire 1101 comprising the first expansion feature 1109. When the frame 403 moves from the radially-contracted second position (e.g., illustrated in FIG. 13) to the radially-expanded first position (e.g., illustrated in FIG. 12), the first expansion feature 1109 can elongate and straighten, thus facilitating expansion of the frame 403. In aspects, the first attachment wire 1101 can comprise a shape memory that can allow the first expansion feature 1109 to revert back to the shape illustrated in FIG. 13. For example, in aspects, the first attachment wire 1101 may include or be manufactured from a shape-memory material.

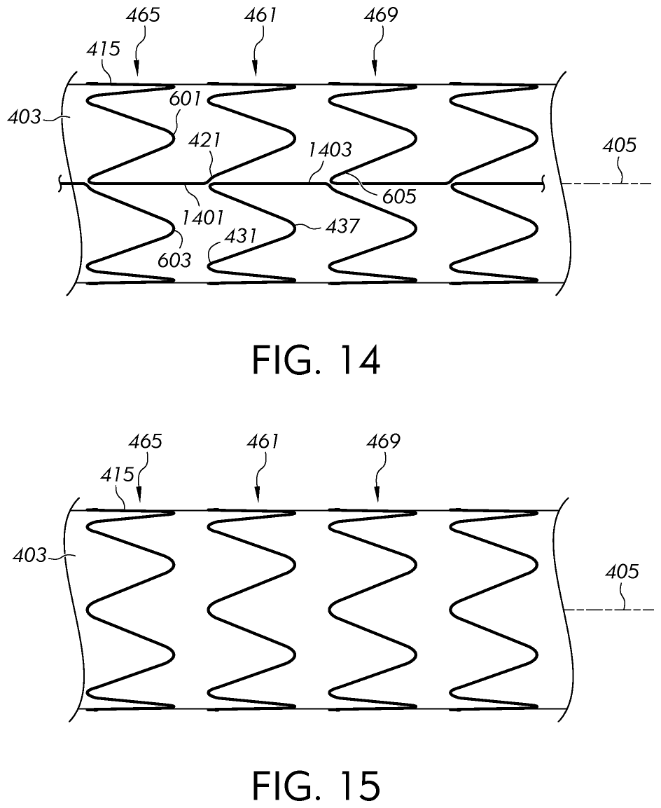

FIGS. 14-15 illustrate another example aspect of forming the frame 403, wherein the wire 415 can comprise a single, continuous wire. For example, the wire 415 can extend continuously to form the first peak portion 421, the second peak portion 431, the valley portion 437, the first axially-offset valley portion 601, the second axially-offset valley portion 603, and the first axially-offset peak portion 605. In aspects, the wire 415 can be wound circumferentially around the frame axis 405 to form the second level 465. Upon winding about 360 degrees around the frame axis 405 to form the second level 465, the wire 415 can comprise a first connection portion 1401 that extends from the second level 465 along the frame axis 405 toward the first level 461. The first connection portion 1401 can comprise the portion of the wire 415 that connects the second level 465 to the first level 461. In aspects, the first connection portion 1401 can extend substantially parallel to the frame axis 405, though, the first connection portion 1401 can extend in a non-parallel direction relative to the frame axis 405. In aspects, the first connection portion 1401 can intersect a peak portion of the first level 461 such that the first connection portion 1401 can form a part of the peak portion. The wire 415 can be wound about 360 degrees around the frame axis 405 beginning at the first connection portion 1401 to form the first level 461.

Upon winding about 360 degrees around the frame axis 405 to form the first level 461, the wire 415 can comprise a second connection portion 1403 that extends from the first level 461 along the frame axis 405 toward the third level 469. The second connection portion 1403 can comprise the portion of the wire 415 that connects the first level 461 to the third level 469. In aspects, the second connection portion 1403 can extend substantially parallel to the frame axis 405, though, the second connection portion 1403 can extend in a non-parallel direction relative to the frame axis 405. In aspects, the first connection portion 1401 and the second connection portion 1403 can extend substantially co-axially. In aspects, the second connection portion 1403 can intersect a peak portion of the third level 469 such that the second connection portion 1403 can form a part of the peak portion. The wire 415 can continue to be wound around the frame axis 405 in a similar manner, with connection portions connecting adjacent levels of the frame 403. In this way, a single wire 415 can be continuously wound around the frame axis 405 to form the frame 403. FIG. 14 illustrates a front side of the frame 403, in which the connection portions 1401, 1403 are located, while FIG. 15 illustrates an opposing rear side of the frame 403, in which the connection portions 1401, 1403 are obstructed from view. As such, the connection portions 1401, 1403 may be located on one side of the frame 403. In aspects, the waveform shape of the single continuous wire 415 of FIGS. 14-15 can comprise a continuous wave (e.g., as illustrated in FIGS. 10-15) or the shape illustrated in FIGS. 4-9 with peak portions and valley portions of differing amplitudes The frame 403 comprising the single continuous wire 415 can provide several benefits, for example, by improving the ease of manufacturability of the wire 415 because a single wire can be used.

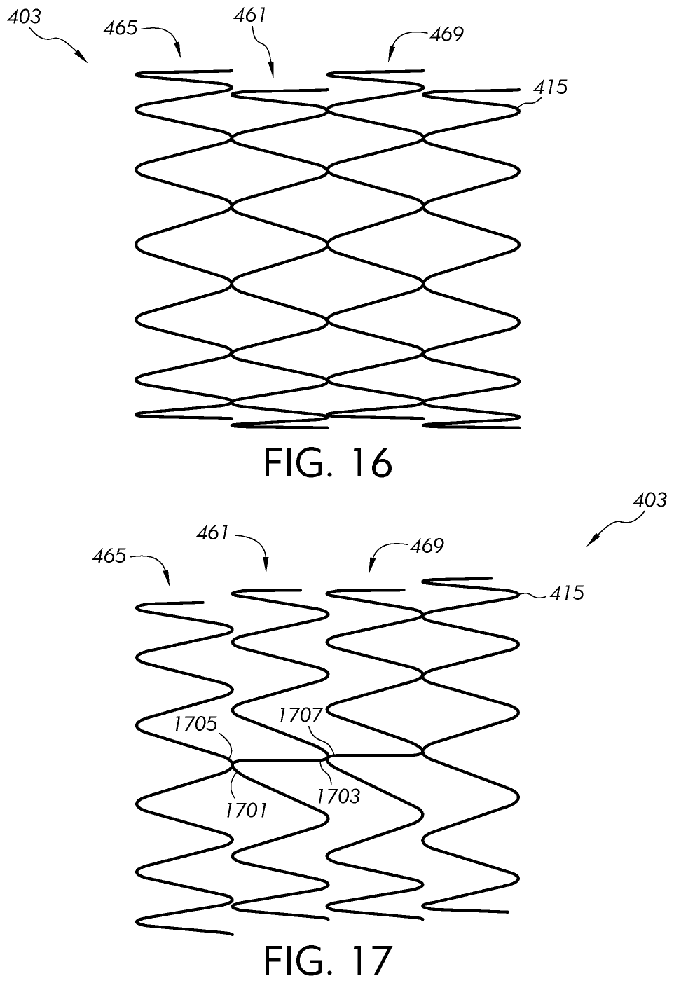

FIG. 16 illustrates additional example aspects of forming the frame 403, wherein the wire 415 can comprise a single, continuous wire. In contrast to the single continuous wire 415 of FIGS. 14-15 in which the different levels 461, 465, 469 are spaced-apart and separated, with the connection portions 1401, 1403 extending between the levels 461, 465, 469, the single continuous wire 415 of FIG. 16 can comprise attached peak portions and valley portions. For example, with reference to the first level 461, each of the peak portions in the first level 461 is attached to an axially-offset valley portion of the second level 465. Likewise, each of the valley portions in the first level 461 is attached to an axially-offset peak portion of the third level 469. In this way, each level 461, 465, 469 is attached at a plurality of locations to adjacent levels on both sides along the frame axis 405.

FIG. 17 illustrates additional example aspects of forming the frame 403, wherein the wire 415 can comprise a single, continuous wire. In contrast to the single continuous wire 415 of FIGS. 14-15 in which the different levels 461, 465, 469 are spaced-apart and separated, and in contrast to the single continuous wire 415 of FIG. 16 in which the different levels are attached at a plurality of locations to adjacent levels, the single continuous wire 415 of FIG. 17 can comprise one peak portion of a level attached to an axially-offset valley portion and one valley portion of the level attached to an axially-offset peak portion. For example, the first level 461 can comprise a peak portion 1701 attached to a valley portion 1703. The peak portion 1701 can be attached to an axially-offset valley portion 1705 of the second level 465. The valley portion 1703 can be attached to an axially-offset peak portion 1707 of the third level 469. In aspects, zero other peak portions of the first level 461 may be attached to the second level 465, and zero other valley portions of the first level 461 may be attached to the third level 469. Accordingly, in aspects, within each level, one peak portion may be attached and one valley portion may be attached.

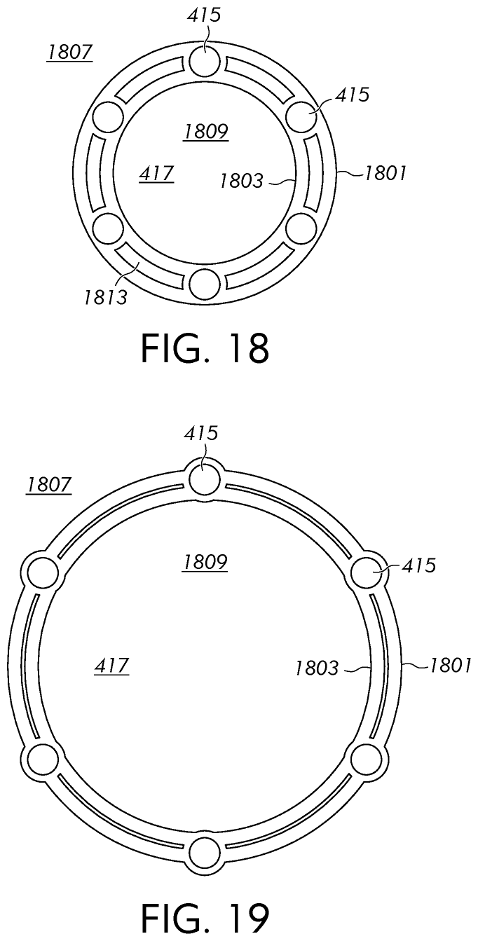

FIGS. 18-19 illustrate a cross-sectional view of the sheath 401 in which the sheath 401 can comprise a cover 1801 and a jacket 1803 that can cover a portion of the wire 415. For example, the cover 1801 can extend along the frame axis 405 and can cover an outer radial side 1807 of the wire 415 opposite the lumen 417. In aspects, the cover 1801 can contact an outer radial surface of the wire 415 and may be attached or adhered to the outer radial surface. In aspects, the cover 1801 can comprise one or more of an expanded polytetrafluoroethylene, a thermoplastic polyurethane, silicone, elastane, a thermoplastic polyolefin, or a thermoplastic elastomer. The jacket 1803 can extend along the frame axis 405 and may cover an inner radial side 1809 of the wire 415. In this way, the wire 415 may be radially positioned between the cover 1801 at the outer radial side 1807 and the jacket 1803 at the inner radial side 1809. The jacket 1803 can comprise, for example, one or more of a polyester fabric material or a polymeric material. The cover 1801 and the jacket 1803 can be attached to the wire 415 in several ways. For example, in aspects, one or more of the cover 1801 or the jacket 1803 can be sewn to the wire 415, though, in other aspects, the cover 1801 or the jacket 1803 can be applied as a liquid and cured. In aspects, the cover 1801 and/or the jacket 1803 can be applied to the wire 415 (e.g., which may comprise a nitinol material) while the wire 415 is at the radially-expanded position of FIG. 19, and then the cover 1801, the jacket 1803, and the wire 415 can be moved from the radially-expanded position to the radially-contracted position by a shape-memory heat setting of the wire 415. In the radially-contracted second position (e.g., illustrated in FIG. 18), a gap 1813 may be defined between the cover 1801 and the jacket 1803, with the gap 1813 present between portions of the wire 415. As the sheath 401 expands to the radially-expanded first position (e.g., illustrated in FIG. 19), the gap 1813 may radially shrink and be reduced in size, such that an inner radial surface of the cover 1801 and an outer radial surface of the jacket 1803 may be in closer proximity or touching, such that the gap 1813 may not be present. In this way, the gap 1813 can facilitate expansion and contraction of the sheath 401. The selection of material of the cover 1801 and the jacket 1803 can protect the vasculature as the sheath 401 is moved to the desired location within the vasculature of a patient, for example, by forming a smooth outer surface that will not catch on vessel walls and will traverse within the vasculature with less applied force and greater predictability.

In aspects, in the radially-collapsed position, an inner diameter of the jacket 1803 can be within a range from about 2 millimeters (“mm”) to about 7 mm, from about 4 mm to about 5 mm, or about 4.5 mm. In aspects, in the radially-collapsed position, an outer diameter of the cover 1801 can be within a range from about 5 mm to about 9 mm, from about 6 mm to about 8 mm, or about 7 mm. In aspects, in the radially-expanded position, an inner diameter of the jacket 1803 can be within a range from about 10 millimeters (“mm”) to about 14 mm, from about 11 mm to about 13 mm, or about 12 mm. In aspects, in the radially-expanded position, an outer diameter of the cover 1801 can be within a range from about 11 millimeters (“mm”) to about 15 mm, from about 12 mm to about 14 mm, or about 13 mm.

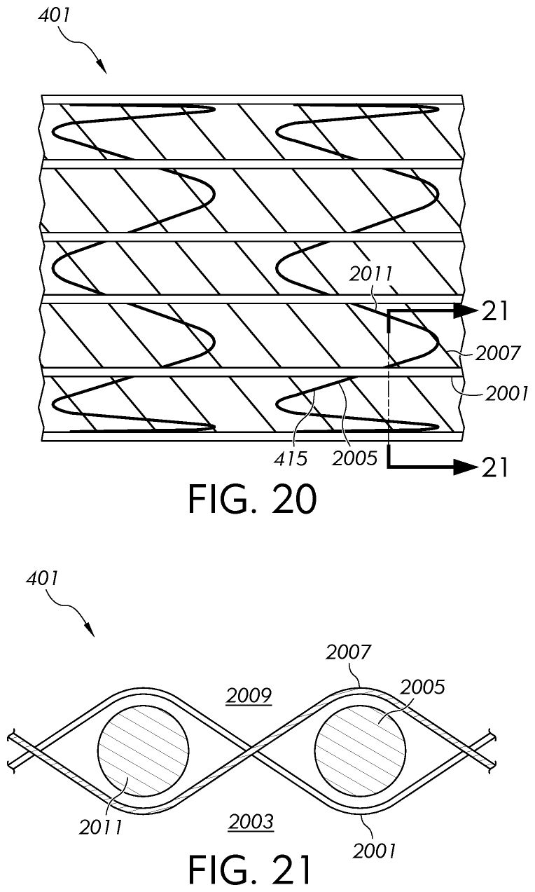

FIGS. 20-21 illustrate the sheath 401 comprising the wire 415, wherein the frame 403 can comprise a braided shape. FIG. 20 illustrates a side view of the sheath 401 and FIG. 21 illustrates a cross-sectional view of a portion of the sheath 401 along lines 21-21 of FIG. 20. In aspects, the braided shape can comprise a first braided portion 2001 positioned at an inner radial side 2003 of a first wire portion 2005 of the wire 415. The frame 403 can comprise a second braided portion 2007 positioned at an outer radial side 2009 of the first wire portion 2005. In aspects, at a different location, the first braided portion 2001 can be positioned at the outer radial side 2009 of a second wire portion 2011 of the wire 415 and the second braided portion 2007 can be positioned at the inner radial side 2003 of the second wire portion 2011. In this way, the first braided portion 2001 and the second braided portion 2007 can be interlaced and wrapped around wire portions 2005, 2011 of the wire 415, such that the first braided portion 2001 and the second braided portion 2007 can alternate positions between the inner radial side 2003 and the outer radial side 2009. For example, the first wire portion 2005 can comprise a strand or a segment of the wire 415, and the second wire portion 2011 can comprise a different strand or segment of the wire 415. The wire portions 2005, 2011 can be positioned radially between the braided portions 2001, 2007. For example, the first braided portion 2001 can move from the inner radial side 2003 of the first wire portion 2005 to the outer radial side 2009 of the second wire portion 2011 while the second braided portion 2007 can move from the outer radial side 2009 of the first wire portion 2005 to the inner radial side 2003 of the second wire portion 2011. Additionally, other wire portions can be surrounded in a substantially identical manner, with the first braided portion 2001 and the second braided portion 2007 alternating positions between the inner radial side 2003 and the outer radial side 2009. In aspects, the cover 1801 and the jacket 1803 can be positioned covering the braided portions 2001, 2007, with the cover 1801 covering the outer radial side 2009 (e.g., attached to an outer radial surface of the braided portions 2001, 2007) and the jacket 1803 covering the inner radial side 2003 (e.g., attached to an inner radial surface of the braided portions 2001, 2007).

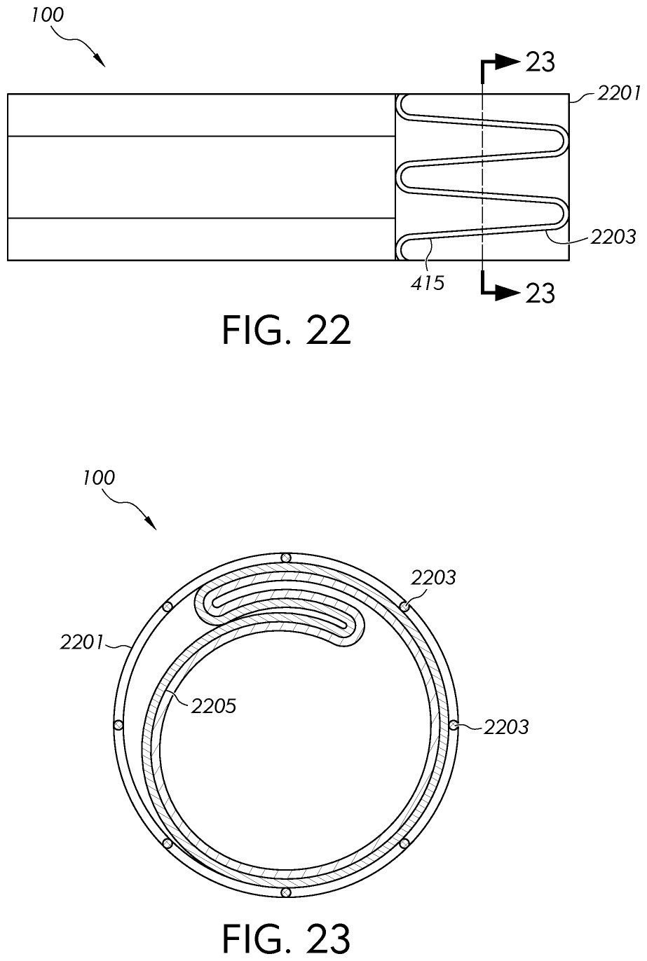

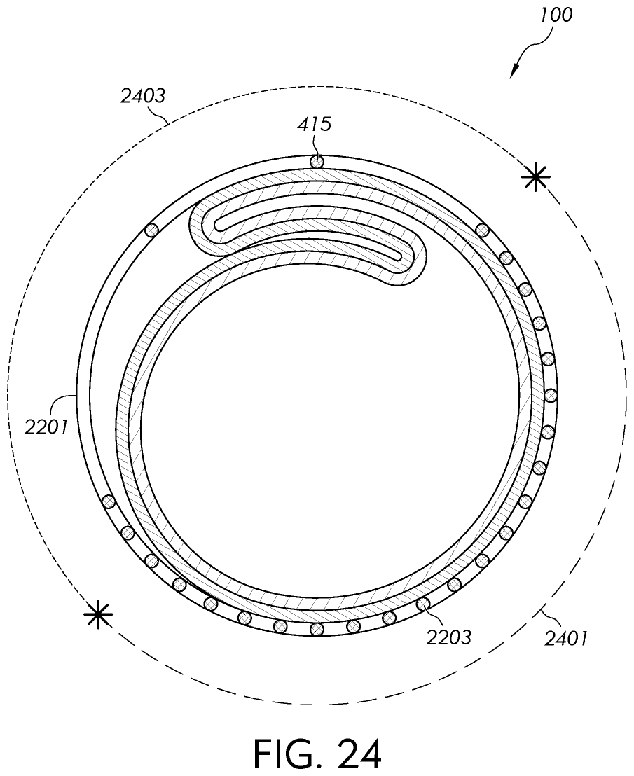

FIG. 22 illustrates a side view of an end of the transcatheter heart valve implant apparatus 100 and FIG. 23 illustrates a cross-sectional view as viewed along lines 23-23 of FIG. 22. Referring to FIG. 22, in aspects, the wire 415 can extend to a tip 2201 of the transcatheter heart valve implant apparatus 100. For example, an end portion 2203 of the wire 415 can be embedded in the tip 2201. The tip 2201 can comprise an elastic material within which the end portion 2203 of the wire 415 can be embedded. The wire 415 can comprise the waveform shape, for example, the continuous or non-continuous waveform shape illustrated and described relative to FIGS. 4-21. The wire 415 can provide rigidity to the tip 2201 to limit the likelihood of the tip 2201 folding or bending in an unintended manner during movement within the vasculature. In aspects, a valve 2205 can be positioned at least partially within the tip 2201. In aspects, for example, as illustrated in FIG. 23, the end portion 2203 of the wire 415 can be evenly spaced circumferentially about the tip 2201. However, as illustrated in FIG. 24, in aspects, the end portion 2203 of the wire 415 may not be evenly spaced circumferentially about the tip 2201. Rather, the end portion 2203 can comprise a greater pitch, and, thus, a higher frequency, at a first region 2401 of the tip 2201, and a lesser pitch, and, thus, a lower frequency, at a second region 2403 of the tip 2201. In this way, the end portion 2203 can comprise a non-constant circumferential spacing about the tip 2201. A benefit of this configuration is that the end portion 2203 of the wire 415 may be provided with an increased pitch in a tip region, for example, the first region 2401, that may not expand, thus providing extra support to the first region 2401 while allowing the second region 2403 to radially expand.

By providing sheaths that comprise a non-fixed diameter, for example, by being radially-expandable and radially-contractable, the sheaths disclosed herein can reduce the likelihood of damage within the vasculature while allowing relatively large valves to be received within the lumen of a sheath. Further, the sheath can radially-expand when a valve is received, and then radially-contract after the valve is delivered, thus reducing stress on a vessel wall. Further, the wire, jacket, and cover can provide radial strength to the sheath as a delivery system is passed through the sheath. In addition, due to the sheath comprising a variable cross-sectional size, the sheath can be used in a variety of applications across a number of therapy areas with valves and prostheses of various sizes. Additionally, in aspects, the sheaths can be manufactured in several ways. For example, the sheath can comprise a single wire or a plurality of wires. Further, based on the strength and flexibility requirements, the sheath can comprise different waveform designs and/or peak portions and valley portions that may or may not be attached. In aspects, the wire can be laser patterned and formed, such that when the sheath comprises a single continuous wire, the wire may be a one-piece formed material.

It should be understood that while various aspects have been described in detail relative to certain illustrative and specific examples thereof, the present disclosure should not be considered limited to such, as numerous modifications and combinations of the disclosed features are possible without departing from the scope of the following claims.

What is claimed is:

1. A transcatheter heart valve implant apparatus comprising:
a frame extending along a frame axis and radially expandable between a first position, in which the frame comprises a first cross-sectional size, and a second position, in which the frame comprises a second cross-sectional size different than the first cross-sectional size, the frame comprising:
a wire extending circumferentially around the frame axis to define a lumen, the wire comprising a first peak portion comprising a first amplitude measured from a plane that is perpendicular to the frame axis and intersects the wire, a second peak portion comprising a second amplitude measured from the plane, and a valley portion attaching the first peak portion and the second peak portion, wherein the first amplitude is different than the second amplitude, and the first peak portion and the second peak portion are positioned on a first side of the plane and the valley portion is positioned on an opposing second side of the plane; and
a cover extending along the frame axis and covering an outer radial side of the wire opposite the lumen.

2. The transcatheter heart valve implant apparatus of claim 1, wherein the first peak portion is attached to a first axially-offset valley portion of the wire at a first attachment location, the second peak portion is attached to a second axially-offset valley portion of the wire at a second attachment location, and the valley portion is attached to a first axially-offset peak portion of the wire at a third attachment location.

3. The transcatheter heart valve implant apparatus of claim 2, wherein the wire extends continuously to form the first peak portion, the second peak portion, the valley portion, the first axially-offset valley portion, the second axially-offset valley portion, and the first axially-offset peak portion.

4. The transcatheter heart valve implant apparatus of claim 1, wherein the frame further comprises an attachment wire extending in an axial direction substantially parallel to the frame axis, the attachment wire attached to a plurality of different locations of the wire.

5. The transcatheter heart valve implant apparatus of claim 1, wherein the cover comprises one or more of an expanded polytetrafluoroethylene, a thermoplastic polyurethane, silicone, elastane, a thermoplastic polyolefin, or a thermoplastic elastomer.

6. The transcatheter heart valve implant apparatus of claim 1, further comprising a jacket extending along the frame axis and covering an inner radial side of the wire.

7. The transcatheter heart valve implant apparatus of claim 6, wherein the jacket comprises one or more of a polyester fabric material or a polymeric material.

8. The transcatheter heart valve implant apparatus of claim 1, wherein the wire comprises one or more of nickel titanium or stainless steel.

9. The transcatheter heart valve implant apparatus of claim 1, wherein the frame comprises a braided shape with a first braided portion positioned at an inner radial side of a first wire portion of the wire and a second braided portion positioned at an outer radial side of the first wire portion, and the first braided portion positioned at the outer radial side of a second wire portion of the wire and the second braided portion positioned at the inner radial side of the second wire portion.

10. The transcatheter heart valve implant apparatus of claim 1, wherein an end portion of the wire is embedded in a tip of the transcatheter heart valve implant apparatus.

11. A transcatheter heart valve implant apparatus comprising:
a frame extending along a frame axis and radially expandable between a first position, in which the frame comprises a first cross-sectional size, and a second position, in which the frame comprises a second cross-sectional size different than the first cross-sectional size, the frame comprising:
a wire extending circumferentially around the frame axis to define a lumen, the wire comprising:
a first peak portion comprising a first amplitude measured from a plane that is perpendicular to the frame axis and intersects the wire, the first peak portion attached to a first axially-offset valley portion of the wire at a first attachment location;
a second peak portion comprising a second amplitude measured from the plane, the second peak portion attached to a second axially-offset valley portion of the wire at a second attachment location; and
a valley portion attaching the first peak portion and the second peak portion, the valley portion attached to a first axially-offset peak portion of the wire at a third attachment location, wherein the first amplitude is different than the second amplitude, and the first attachment location, the second attachment location, and the third attachment location are axially-offset along the frame axis; and
a cover extending along the frame axis and covering an outer radial side of the wire opposite the lumen.

12. The transcatheter heart valve implant apparatus of claim 11, wherein the wire extends continuously to form the first peak portion, the second peak portion, the valley portion, the first axially-offset valley portion, the second axially-offset valley portion, and the first axially-offset peak portion.

13. The transcatheter heart valve implant apparatus of claim 11, wherein the frame further comprises an attachment wire extending in an axial direction substantially parallel to the frame axis, the attachment wire attached to a plurality of different locations of the wire.

14. The transcatheter heart valve implant apparatus of claim 11, wherein the cover comprises one or more of an expanded polytetrafluoroethylene, a thermoplastic polyurethane, silicone, elastane, a thermoplastic polyolefin, or a thermoplastic elastomer.

15. The transcatheter heart valve implant apparatus of claim 11, further comprising a jacket extending along the frame axis and covering an inner radial side of the wire, the jacket comprising one or more of a polyester fabric material or a polymeric material.

16. The transcatheter heart valve implant apparatus of claim 11, wherein the frame comprises a braided shape with a first braided portion positioned at an inner radial side of a first wire portion of the wire and a second braided portion positioned at an outer radial side of the first wire portion, and the first braided portion positioned at the outer radial side of a second wire portion of the wire and the second braided portion positioned at the inner radial side of the second wire portion.

17. The transcatheter heart valve implant apparatus of claim 11, wherein an end portion of the wire is embedded in a tip of the transcatheter heart valve implant apparatus.

18. A transcatheter heart valve implant apparatus comprising:
a frame extending along a frame axis and radially expandable between a first position, in which the frame comprises a first cross-sectional size, and a second position, in which the frame comprises a second cross-sectional size different than the first cross-sectional size, the frame comprising:
a first wire portion extending circumferentially around the frame axis to define a first portion of a lumen, the first wire portion comprising a waveform shape comprising a plurality of first peak portions and a plurality of first valley portions;
a second wire portion extending circumferentially around the frame axis to define a second portion of the lumen, the second wire portion comprising the waveform shape comprising a plurality of second peak portions and a plurality of second valley portions, the first wire portion axially offset from the second wire portion along the frame axis; and
an attachment wire extending along the frame axis and attached at a first end to the first wire portion and at a second end to the second wire portion; and
a cover extending along the frame axis and covering an outer radial side of the wire opposite the lumen.

19. The transcatheter heart valve implant apparatus of claim 18, wherein the cover comprises one or more of an expanded polytetrafluoroethylene, a thermoplastic polyurethane, silicone, elastane, a thermoplastic polyolefin, or a thermoplastic elastomer.

20. The transcatheter heart valve implant apparatus of claim 18, further comprising a jacket extending along the frame axis and covering an inner radial side of the wire, the jacket comprising one or more of a polyester fabric material or a polymeric material.
